(19) United States
(12) Patent Application Publication
(10) Pub. No.: US 2023/0325901 A1
(43) Pub. Date: Oct. 12, 2023

(54) CANNABIS MACHINE LEARNING-BASED RECOMMENDATION SYSTEM

(71) Applicant: Symphony9 LLC, Glenview, IL (US)

(72) Inventors: Santoash Rajaram, Portland, OR (US); Karan Mishra, Glenview, IL (US)

(21) Appl. No.: 18/131,149

(22) Filed: Apr. 5, 2023

Related U.S. Application Data
(60) Provisional application No. 63/362,593, filed on Apr. 6, 2022.

(51) Int. Cl. G06Q 30/0601; G06N 3/08; G06N 3/045; G06V 40/20; G06V 40/16; G06V 10/82

(52) U.S. Cl. CPC G06Q 30/0631; G06N 3/08; G06V 10/82; G06V 40/20; G06V 40/176; G06N 3/045

(57) ABSTRACT

User data is collected regarding the user's lifestyle, preferences, and medical conditions and used to recommend a cannabis product. The collected user data is clustered into a group with user data from other users to create a user profile, and a package of cannabis products is created and provided to the user based on the user profile. A recommendation engine recommends a strain of cannabis product within the package to the user based on a purpose or occasion provided by the user for the cannabis consumption. To evaluate the effectiveness of the recommendation for the given purpose or occasion, pre-consumption and post-consumption video data of the user is captured and processed by a deep learning system for the selected strain. Feedback data is also collected from the user to feed back to fine tune the clustering and the contents of the package recommended for the user profile.

### CROSS-REFERENCE TO RELATED APPLICATIONS

The present application claims priority to U.S. Provisional Patent Application No. 63/362,593, filed Apr. 6, 2022, entitled “Cannabis Recommendation System.” The contents of that patent application are hereby incorporated by reference.

### TECHNICAL FIELD

This disclosure describes workflows and machine learning models for training a machine learning recommendation model to select a cannabis product that is specific to each user based on what has worked for that particular user.

### BACKGROUND

Several researchers recently have shown how essential the endocannabinoid system is for health. Consuming a small dose of cannabis on a regular basis is shown to increase the cannabinoid receptors, the increase of which is shown by an increase in baseline of endocannabinoid activity which in turn acts as a tonic to the central physiologic healing system. However, every individual reacts differently to the substances in a commercially available cannabis strain due to the cannabinoid receptors in their system and external factors like stress level, physical pain, diet, family situation, etc. The variety of available cannabis strains and the variety of reasons for consuming cannabis make selection of the best strains for consumers increasingly difficult. Existing methods for recommending cannabis strains involve making suggestions based on what worked for similar users or based on content similar to the content you liked. Neither of these methods is effective due to each individual's unique endocannabinoid system.

### SUMMARY

Various examples are now described to introduce a selection of concepts in a simplified form that are further described below in the Detailed Description. This Summary is not intended to identify key or essential features of the claimed subject matter, nor is it intended to be used to limit the scope of the claimed subject matter.

The following disclosure describes a method of recommending a cannabis or other medicinal product to a user using a machine learning model. The method described herein improves the cannabis strain selection process by allowing users to conduct personalized experiments to select a cannabis strain that works best for their specific needs. The method includes collecting data from the user regarding at least one of the user's lifestyle, preferences, medical conditions, or a video of the user performing a series of movements, and clustering the collected user data into a group with user data from other users to create a user profile in a machine learning system. Each group of clustered user data may correspond to a different curated box of strains of cannabis products and may determine a design of the package and a marketing message associated with the package. The created user profile may include at least one of favorite strains of cannabis product, reasons for consuming cannabis, answers to lifestyle related questions, or a video of the person doing predetermined movements. A package of cannabis products is created based on the user profile and provided to the user. A strain of cannabis product within the package of cannabis products is recommended to the user by the machine learning system based on a purpose or occasion provided by the user for the cannabis consumption. Pre-consumption and post-consumption video data of the user is captured for a selected strain of cannabis product and processed to measure effectiveness of the selected strain of the cannabis product for the given purpose or occasion. Feedback data from the user is collected regarding the selected strain of cannabis product to feed back to fine tune the machine learning algorithm for adjusting the contents of the package of cannabis products recommended for the user profile.

In sample configurations, recommending to the user a strain of cannabis product within the package of cannabis products based on a purpose or occasion provided by the user for the cannabis consumption may include creating a user vector that captures the user's interests relative to a predetermined number of unique properties of available strains of cannabis product, creating a library matrix of strains of cannabis product that includes the available strains of cannabis product and features that associate each strain of cannabis product with its properties, providing the user vector and the library matrix of cannabis product to an algorithm that creates a ranked list of strains of cannabis product that most closely match the user vector, and providing to the user at least one of the strains of cannabis product in the ranked list of strains in the package of cannabis products. In the sample configurations, an input strain of cannabis product may be provided and similarity scores computed between the input strain and strains in the library matrix. The similarity scores may be used to generate the ranked list with a predetermined number of most similar strains appearing first.

In other sample configurations, a cannabis product delivery service maintains a list of strains of cannabis product to which the user currently has access, updates the list of strains of cannabis product as the user consumes strains of cannabis product, and updates a server for a cannabis product delivery system as the user consumes strains of cannabis product so that the cannabis product delivery system knows when and what to reorder.

In further sample configurations, capturing pre-consumption and post-consumption video data of the user for a selected strain of cannabis product includes taking a pre-consumption video of the user's face a first predetermined number of minutes before consuming the selected strain of cannabis product and taking a post-consumption video of the user's face a second predetermined number of minutes after consuming the selected strain of cannabis product. The pre-consumption and post-consumption video data of the user may be further processed by a deep learning system to measure effectiveness of the selected strain of the cannabis product for the given purpose or occasion by extracting user data from facial cues in the pre-consumption video and post-consumption video.

In still further configurations, collecting feedback data from the user regarding the selected strain of cannabis product to feed back to fine tune the clustering and contents of the package of cannabis products recommended for the user profile may include identifying a plurality of features of the strains of cannabis products that may be used to select a strain for user consumption, prioritizing a feature, and ranking strains of cannabis products available for user consumption based on the prioritized feature. In some examples, a weight may be assigned to the prioritized feature according to a feedback score for the selected strain. The collected feedback data from the user regarding the selected strain of cannabis product may include data relating to tetrahydrocannabinol (THC) content, medicinal use and effects, and flavor and aroma of the selected strain of cannabis product. A final evaluation score for the selected strain of cannabis product may be determined that is a sum of scores relating to the THC content, the medicinal use and effects, and the flavor and aroma of the selected strain of cannabis product.

In the example configurations, a score of THC effectiveness of the selected strain of cannabis product may be determined by processing the pre-consumption and post-consumption video data of the user using a deep learning model. Determining the score of THC effectiveness may include taking a pre-consumption picture of the user's face a first specified time before consumption of the selected strain of cannabis product, consuming a predefined amount of the selected strain of cannabis product, taking a post-consumption picture of the user's face a second specified time after consumption of the selected strain of cannabis product, and providing the pre-consumption picture of the user's face and the post-consumption picture of the user's face to a trained neural network that analyzes the pre-consumption picture of the user's face and the post-consumption picture of the user's face to generate the THC effectiveness score. A medicinal use and effects score may also be determined that is based on effectiveness of the selected strain of cannabis product to treat a particular medicinal case or effect. In addition, feedback data from the user relating to flavor and aroma of the selected strain of cannabis product may be used to determine a flavor and aroma score.

A system is also provided for implementing the method for recommending a cannabis product to a user. The system includes a user profiler that collects data from the user regarding at least one of the user's lifestyle, preferences, medical conditions, or a video of the user performing a series of movements and clusters the collected user data into a group with user data from other users to create a user profile. A strain curator is also provided that creates a package of cannabis products based on the user profile and identifies contents of a package of cannabis products recommended for the user based on the user profile. A delivery service provides the package or cannabis products to the user and tracks a supply of cannabis products available to the user.

In sample configurations, a strain recommender recommends to the user a strain of cannabis product within the package of cannabis products based on a purpose or occasion provided by the user for the cannabis consumption, and a feedback evaluator captures pre-consumption and post-consumption video data of the user for a selected strain of cannabis product and processes the pre-consumption and post-consumption video data of the user to measure effectiveness of the selected strain of the cannabis product for the given purpose or occasion. The feedback evaluator further feeds back data from the user regarding the selected strain of cannabis product to fine tune the clustering by the user profiler and contents of the package of cannabis products recommended for the user profile by the strain curator.

In sample configurations, the strain recommender creates a user vector that captures the user's interests relative to a predetermined number of unique properties of available strains of cannabis product, creates a library matrix of strains of cannabis product that includes the available strains of cannabis product and features that associate each strain of cannabis product with its properties, provides the user vector and the library matrix of cannabis product to an algorithm that creates a ranked list of strains of cannabis product that most closely match the user vector, and recommends inclusion of at least one of the strains of cannabis product in the ranked list of strains in the package of cannabis products.

In other sample configurations, the feedback evaluator collects feedback data from the user regarding the selected strain of cannabis product. The feedback data may include data relating to tetrahydrocannabinol (THC) content, medicinal use and effects, and flavor and aroma of the selected strain of cannabis product. The feedback evaluator may further determine a final evaluation score for the selected strain of cannabis product that is a sum of scores relating to the THC content, the medicinal use and effects, and the flavor and aroma of the selected strain of cannabis product.

A method may be performed by the apparatus, and further features of the method result from the functionality of the apparatus. Also, the explanations provided for each aspect and its implementation apply equally to the other aspects and the corresponding implementations. The different configurations may be implemented in hardware, software, or any combination thereof. Also, any one of the foregoing examples may be combined with any one or more of the other foregoing examples to create a new configuration within the scope of the present disclosure.

### DETAILED DESCRIPTION OF THE DRAWINGS

The foregoing and other beneficial features and advantages of the subject matter described herein will become apparent from the following detailed description in connection with the attached figures, of which:

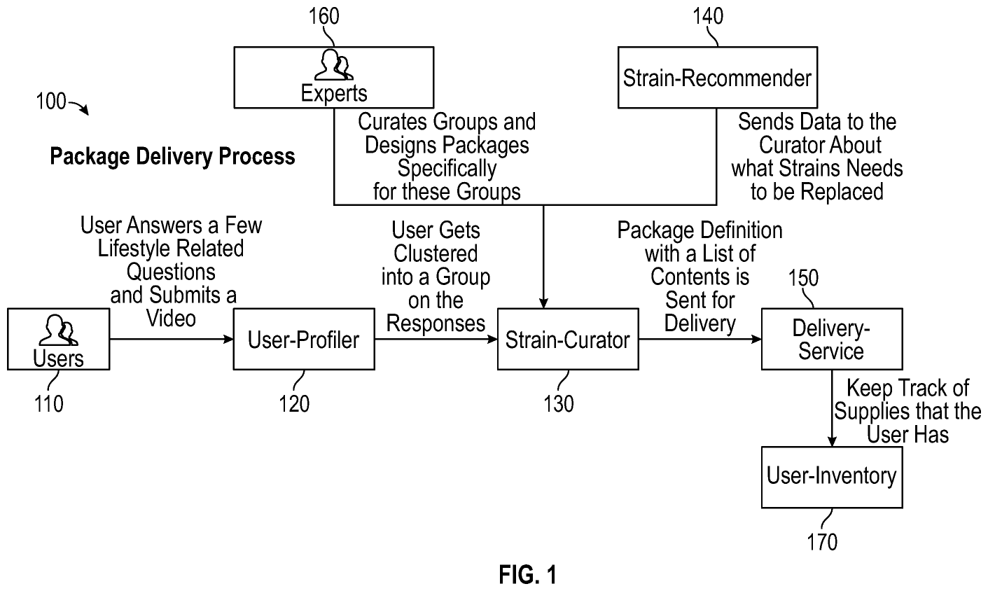

FIG. 1 is a diagram illustrating a cannabis package delivery process in a sample configuration.

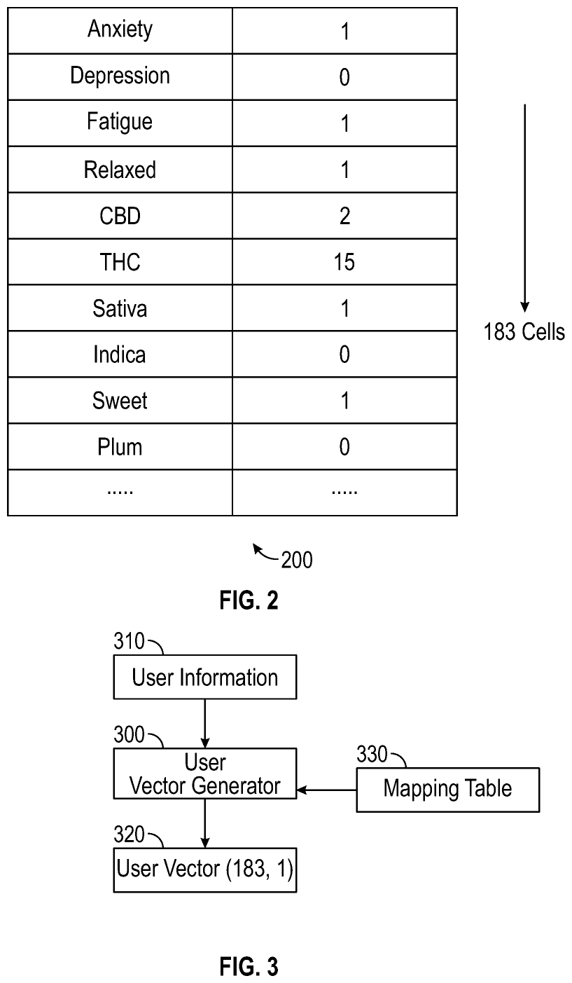

FIG. 2 is a chart illustrating a sample user vector in a sample configuration.

FIG. 3 is a diagram illustrating the generation of a user vector in a sample configuration.

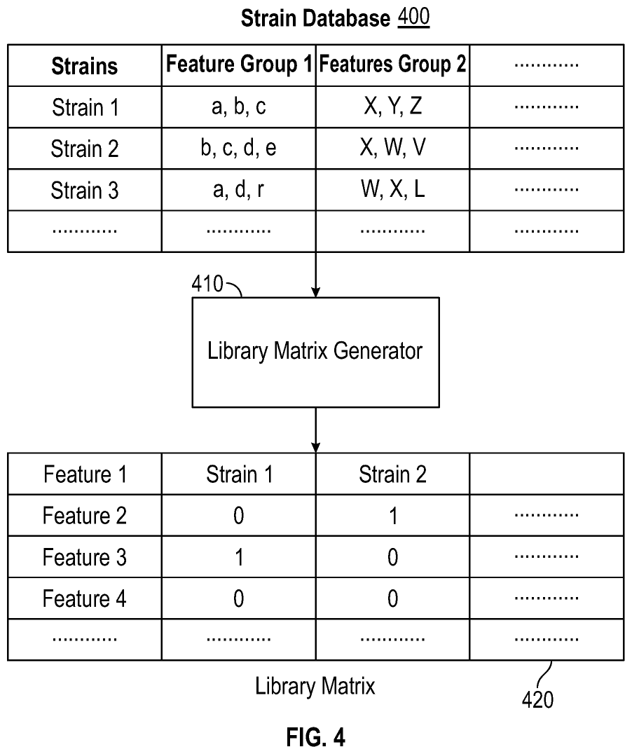

FIG. 4 is a diagram illustrating the generation of a library matrix in a sample configuration.

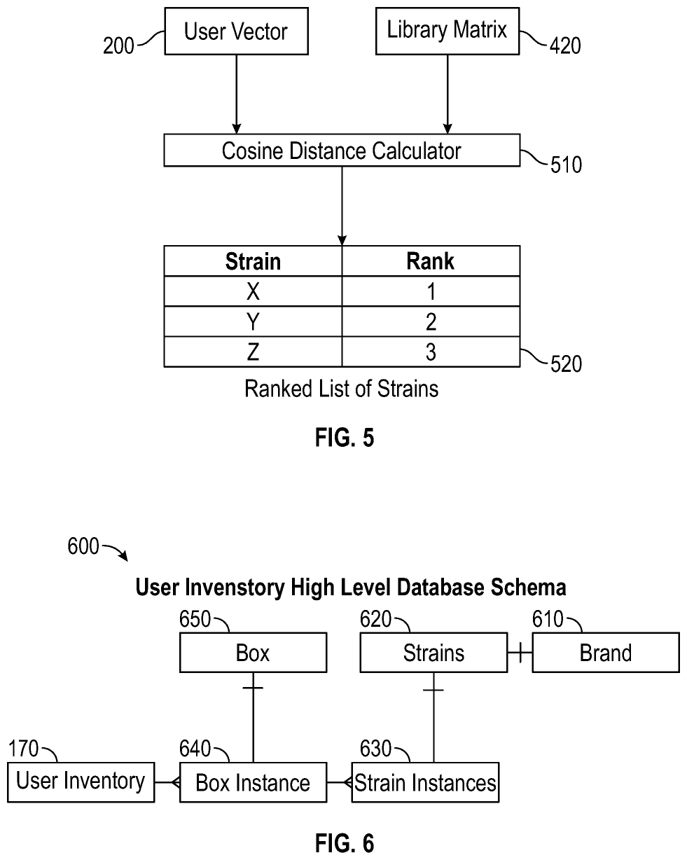

FIG. 5 is a diagram illustrating the generation of recommendations in a sample configuration.

FIG. 6 is a diagram illustrating a user inventory high level database schema in a sample configuration.

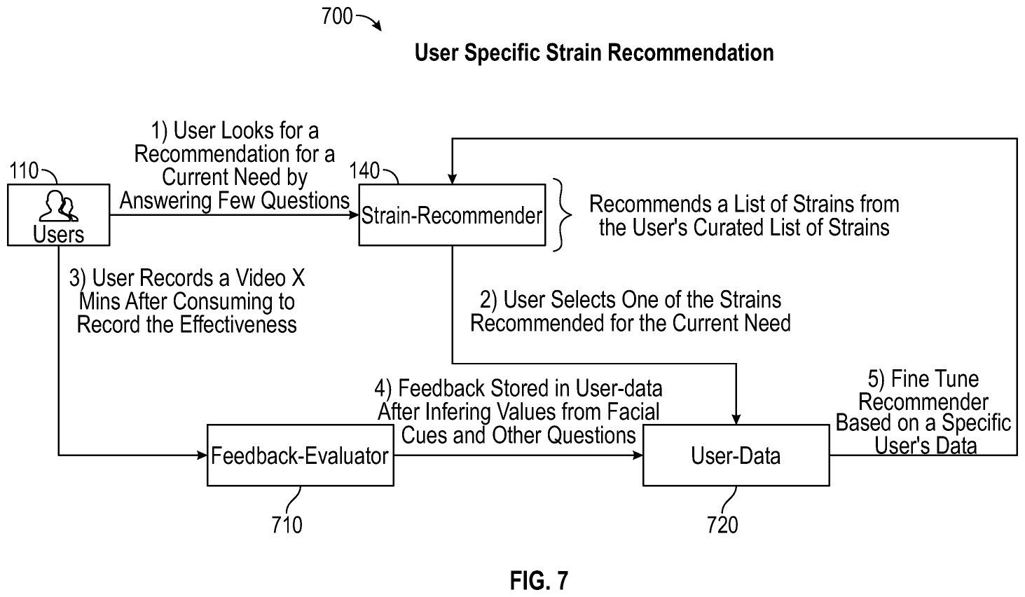

FIG. 7 is a flow diagram of a strain recommendation flow using the strain recommender in a sample configuration.

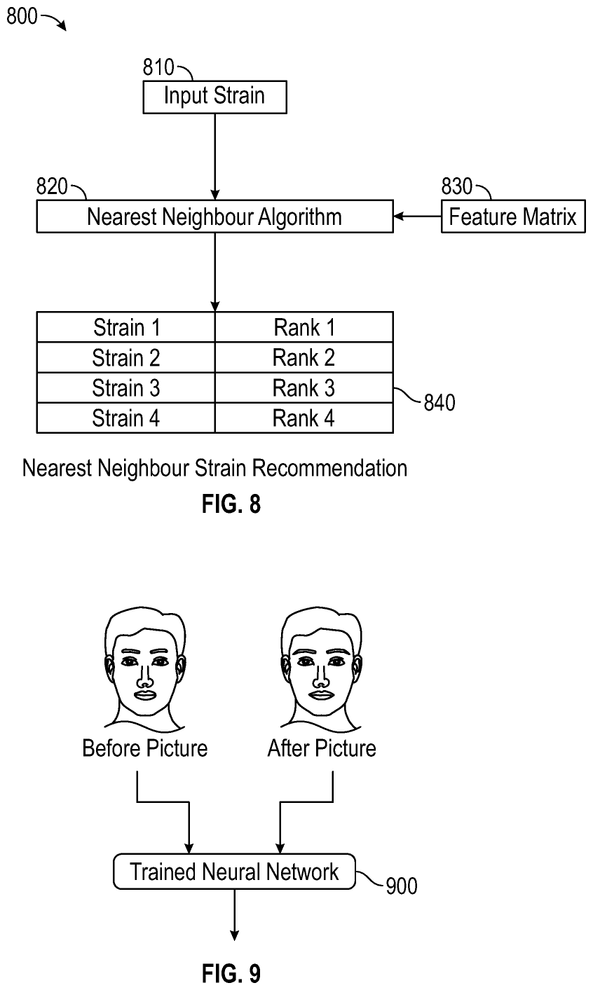

FIG. 8 is a diagram illustrating a nearest neighbor strain recommendation that takes an input strain and uses a nearest neighbor algorithm to select “k” strains from the feature matrix that most closely match the input strain in a sample configuration.

FIG. 9 is a diagram of a THC feedback system in a sample configuration.

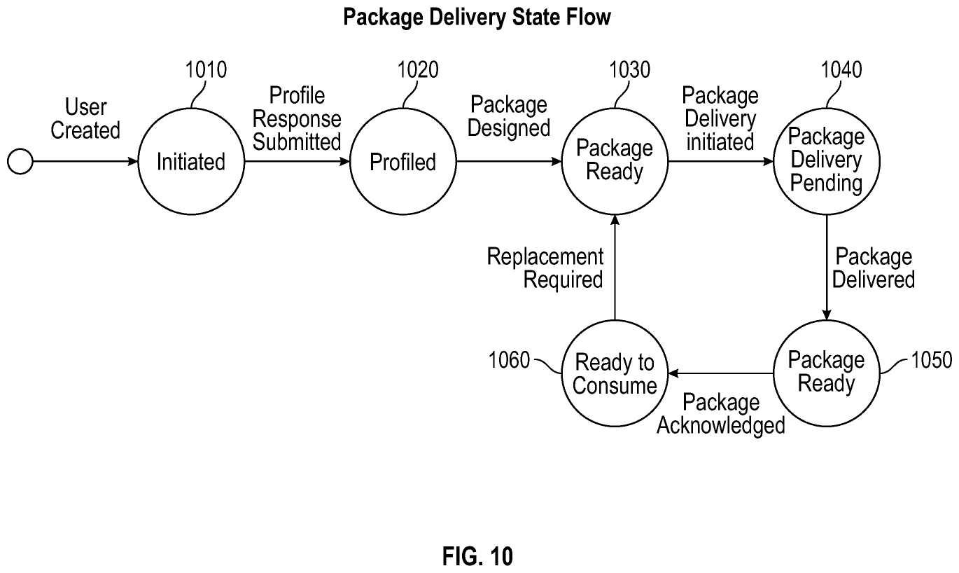

FIG. 10 is a state diagram for tracking users and cannabis packages as the cannabis packages are delivered in a sample configuration.

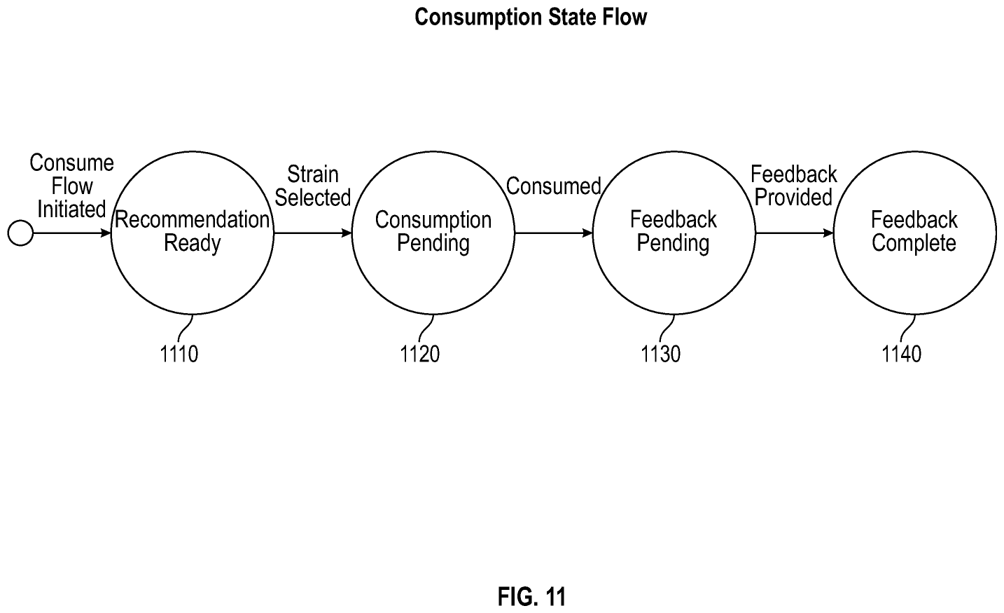

FIG. 11 is a state diagram for tracking consumption of a cannabis product as users consume a strain for a specific reason in a sample configuration.

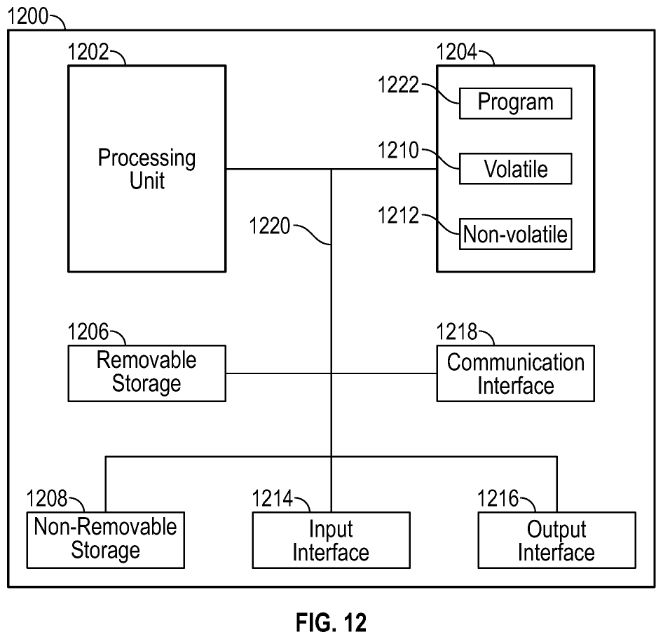

FIG. 12 is a block diagram illustrating circuitry for performing methods and implementing processing features according to the sample configurations.

### DETAILED DESCRIPTION OF ILLUSTRATIVE CONFIGURATIONS

Sample configurations will be described with respect to FIGS. 1-12 for use in selecting a cannabis product, although those skilled in the art will appreciate that the teachings provided herein may be used to select other products. Such configurations are intended to be within the scope of the present disclosure.

As will be described further herein, the cannabis recommendation system provides feedback to update the effectiveness of the machine learning-based recommendation system. For example, a score of tetrahydrocannabinol (THC) effectiveness of the selected strain of cannabis product may be determined by processing the pre-consumption and post-consumption video data of the user using a deep learning model. Determining the score of THC effectiveness may include taking a pre-consumption picture of the user's face a first specified time before consumption of the selected strain of cannabis product, consuming a predefined amount of the selected strain of cannabis product, taking a post-consumption picture of the user's face a second specified time after consumption of the selected strain of cannabis product, and providing the pre-consumption picture of the user's face and the post-consumption picture of the user's face to a trained neural network that analyzes the pre-consumption picture of the user's face and the post-consumption picture of the user's face to generate the THC effectiveness score. A medicinal use and effects score may also be determined that is based on effectiveness of the selected strain of cannabis product to treat a particular medicinal case or effect. In addition, feedback data from the user relating to flavor and aroma of the selected strain of cannabis product may be used to determine a flavor and aroma score. These values may be fed back into the machine learning model to update the recommendation model to provide user optimized recommendations.

Workflows and machine learning models will be described below that may be used to train a machine learning recommendation model specific to each user based on what has worked for that particular user.

### High Level Workflow

Step 1: The user will answer series of question regarding their lifestyle, preferences, and medical conditions and may take a video of themselves performing a series of movements.

Step 2: The data collected from the user will be passed to a machine learning (ML) algorithm (ML Model 1) to cluster the data into a group. A package (combination) of cannabis products will be designed for every group predicted in the system based on the collected user data. Items that are placed into these groups will be statically managed based on user research and eventually assembled dynamically when the user data (ML Model 2) has been obtained.

Step 3: The user receives the assembled cannabis package.

Step 4: Once the user receives the assembled cannabis package, the user can use an app to recommend the right strain within their package for the occasion (ML Model 3). The recommendation will use the user's video and a few simple questions to recommend a list of strains.

Step 5: The user chooses the strain they are planning to consume.

Step 6: The App will remind the user to capture a post-consumption video to measure the effectiveness of the selected strain.

Step 7: Based on the effectiveness of a given strain for a given purpose, the base model will be extended using incremental learning methods specifically adapted for that user (ML Model 4).

Step 8: Feedback data is collected across all users to feed back into the first clustering model (ML Model 1) to fine tune the clustering logic as well as the contents of the package for each cluster.

### Delivery Process

FIG. 1 is a diagram illustrating the cannabis package delivery process 100 in a sample configuration. As illustrated, users 110 answer a few lifestyle related questions and submit a video of themselves performing a series of movements. A user profiler 120 groups the users into clusters based on the users' lifestyles, preferences, reasons for consuming cannabis, etc. Each of these cluster corresponds to a different curated box that will be shipped to the users. The cluster determines a design of the box and the marketing message associated with the box but does not necessarily affect the contents of the box. Some of the features that user profiler 120 will use to profile users may include favorite strains; reasons for consuming cannabis; answers to lifestyle related questions like age range, relationship status, work status, stress level, etc.; video of the person doing guided stretches; etc.

Once a box design and a message to go with the box delivery have been selected, the strain curator 130 selects a list of strains that are to be assembled for the user. This first level of selection of strains is based on a general approximation on what might work for a user based on aggregate behavior without taking into account individual reactions to the strain. This allows the sample size to be reduced to conduct the experiments to narrow down the strain selection even more in later stages. The output of the strain curator 130 is a list of contents to be sent for delivery to the user. The complete box specification includes the name of the curated box (package definition), box type, and list of strains to be included in the box for delivery. For example, a sample box specification may include:

{
“title”: “relax for high stress individuals”,
“message”: “some marketing message”,
“type”: “initial supply”,
“strainIds”: [1231, 2341, 3452, 8903, 8978, 1232],
“mailingAddress”: “1 Infinite loop, Cupertino, CA 95401”
   }

### Recommendation Model

Out of all the possible properties of a cannabis strain, 183 unique properties have been selected to build a machine learning model that may be implemented by the strain curator 130 to recommend the right strains catered to a particular user. The needs and preferences of a user are sent to the machine learning model to recommend the best strains available. The machine learning model checks the user characteristics with the properties of over 2300 strains (and expanding) for recommendation. In a sample configuration, the features of the cannabis strains may be grouped as follows:
Dominant Terpene: 8 Options (Myrcene, Pinene, Limonene, etc.)
Type of Strain: 3 Options (Indica, Sativa and Hybrid)
Medicinal Use Case: 45 Options (Anxiety, Depression, Fatigue, etc.)
Preferred Time of Use: 3 Options (After Work, Day Time, Night Time)
Aroma: 53 Options (Citrus, Pine, Woody, etc.)
Effects: 15 Options (Relaxed, Focused, Giggly, etc.)
Flavor: 51 Options (Sweet, Berry, Spicy, etc.)
Tetrahydrocannabinol (THC) Content: Between Range 0-40
Cannabidiol (CBD) Content: Between Range 0-28
Brands
   This particular set of features can generate the best personalized recommendations by capturing various user needs. This specific set of features not only addresses the medicinal needs of users but also captures preferences such as Aroma and Flavor along with the users' THC thresholds, making the cannabis experience better.

### Step 1: Generation of User Vector

A user vector of length 183 is created that captures the user's interests relative to the 183 unique properties that have been selected to build the recommendation model. The user vector has information of all the curated 183 features that represents the user's interests. The value of a cell is one when that feature is of user interest, zero otherwise (except for THC and CBD which is a natural number within a specified range). FIG. 2 illustrates a sample user vector 200.

FIG. 3 is a diagram illustrating the generation of a user vector in a sample configuration. As illustrated in FIG. 3, a user vector generator 300 implements an algorithm that converts the user information 310 into a user vector 320, while checking the user information with a mapping table 330 for validity and permissible ranges of input. The resulting user vector 320 is the first input to the machine learning model that generates the recommendation. This user vector 320 may be stored in a database to generate recommendations for the user whenever needed, while updating and modifying the user vector 320 to account for changes in user preferences.

### Step 2: Generation of Library Matrix

The library of strains includes the cannabis strains and metadata (features) that associate each strain with its properties. Using this data, a matrix is composed that has rows equal to the number of unique features (183) and columns equal to the total number of strains. This is the second input to the model that generates the recommendation. The matrix is stored in a database to be called whenever required to generate recommendations. The matrix is regenerated whenever the library is updated or modified.

FIG. 4 is a diagram illustrating the generation of a library matrix in a sample configuration. As illustrated, a strain database 400 identifying the respective strains and feature groups is provided to library matrix generator 410, which generates the library matrix 420 having rows for each feature and columns equal to the number of strains.

### Step 3: Generation of Recommendations

The user vector 200 (FIG. 2) and the library matrix 420 are sent to an algorithm that calculates cosine distance for each strain for the given user vector. The distance value may be large for a strain that has less features matching the user vector and small for strains that have more features matching the user vector. The output of the algorithm is a list of strains with the shortest distance listed first. The distance here can be considered as a rank with strains having the shortest distance assigned the least rank. The list obtained from the model can later be filtered/sorted according to the application requirement.

FIG. 5 is a diagram illustrating the generation of recommendations in a sample configuration. As illustrated, the user vector 200 (FIG. 2) and the library matrix 420 are provided to a cosine distance calculator 510 to generate a ranked list of strains 520. It will be appreciated that instead of cosine distance calculation a weight may be added to certain features to prioritize some features over other features.

Referring back to FIG. 1, the strain curator 130 also receives messages from a strain recommender 140 regarding what strains need to be replaced. When these messages from the strain recommender 140 are received, the strain curator 130 figures out the box type and other delivery preferences of this user and sends the package to the delivery service 150 after receiving an approval from the user. From time to time, the experts 160 designing the curated boxes can determine a new strain that might be effective for a specific group of users. In those cases, a recommendation is made to the user to include a new strain in the box. After the approval from the user, the new strains may be sent to the user as part of the package that is delivered by the delivery service 150. All strains delivered to the users are tracked in the user inventory 170.

The delivery service 150 receives the box specification from the strain curator 130 and handles the assembly and the delivery workflow associated with delivering the box to the individual. This delivery service 150 is responsible for updating the user's inventory 170 once delivered. Delivery service 150 is configured with different kinds of box styles that will be supported for different groups. An internal application (FIG. 10) manages this workflow. This internal application tracks all the packages that are to be assembled, packaged, and shipped as well as the tracking information for that shipment. The delivery service 150 may be integrated with mailing APIs like Shippo, USPS, Fedex, etc. to create a shipment once the package has been assembled.

The user inventory 170 maintains a list of the strains to which the user currently has access. The user inventory 170 is useful to start the next level of experimentation and narrowing down based on the consumption pattern and the effectiveness of individual strains for a specific user. As the user consumes strains, their inventory may be updated to a server for the product delivery system so that the product delivery system knows when/what to reorder. This service only has access to what strains a user currently has at their disposal, not the actual usage data.

FIG. 6 is a diagram illustrating a user inventory high level database schema 600 in a sample configuration. As illustrated in FIG. 6, the brand 610 is provided in strains 620 and strain instances 630 that are combined into a box instance 640 including a box 650. The box instances 640 and the associated elements become part of the user inventory 170.

In sample configurations, strain recommender 140 is an ML model that is delivered to a user's device and further tuned on the device based on the feedback collected from the device. The strain recommender 140 is used to identify the strains that work for a given user from their inventory 170. The strain recommender 140 identifies different strains from the user inventory 170 that might work for a user specific need. This identification process is tracked as an experiment with the effectiveness of each strain. The strain recommender 140 maintains different experiments and corresponding reasons for that experiment. For instance, if a user is consuming to relax after a stressful day, that would be tracked as a different experiment compared to the same user consuming to get some sleep.

FIG. 7 is a flow diagram of a strain recommendation flow 700 using strain recommender 140 in a sample configuration. As illustrated, users 110 request a recommendation for a current need by answering a few questions. The strain recommender 140 receives the answers and recommends a list of strains from the user's curated list of strains. The user selects one of the strains recommended by the strain recommender 140 for the current need. The user then consumes the product and records a video a set number of minutes after consuming the product to record the effectiveness of the selected strain. The feedback evaluator 710 evaluates the feedback to extract user data 720 from facial cues and other information in the video as well as inferring values from the user's answers to feedback questions. The specific user data 720 so extracted may then be used to fine tune the recommendations by the strain recommender 140.

In sample configurations, the strain recommender 140 implements a model that generates a list of strains that are similar to the input strain. A Nearest Neighbor algorithm may be trained on the strain feature library that computes a similarity score between the input strain and all the strains in the strain feature library. The output of the model is a list of strains with similar strains appearing first. The model recommends “k” most similar to the input strains where k is an adjustable parameter in the model design.

The process of creating the feature matrix is similar to that of creating the library feature matrix in step 2 of the personalized recommendation model as described above with respect to FIG. 4. However, the feature matrix model for the strain recommender 140 may also accept three additional features from the library database, including Indica Percentile, Sativa Percentile, and Ratings. The feature matrix (e.g., feature matrix 420 in FIG. 4) has rows equal to the number of total unique features and number of columns equal to total number of strains. The Nearest Neighbor model trains on this matrix to generate recommendations.

For example, FIG. 8 is a diagram illustrating a nearest neighbor strain recommendation 800 that takes an input strain 810 and uses a nearest neighbor algorithm 820 to select “k” strains from the feature matrix 830 that most closely match the input strain 810 to provide the nearest neighbor strain recommendations 840. The model may also use a “Brute Force” algorithm to compute a similarity score based on the “Minkowski” metric. The list 840 obtained from the model can be further filtered/sorted according to the application requirements.

The recommended strain(s) is then consumed as part of an experiment that provides useful feedback to the strain recommender 140. In a sample configuration, the methodology for the experiment includes identifying 3-4 features that may be used to narrow down the strains. A recommendation algorithm takes a feature to prioritize and ranks the items in the user's inventory. For each one of those features, a strain is selected from the user's inventory that is scored highly for this feature. This score may be calculated using the recommendation algorithm optimized for that feature. The feedback score is calculated to compute the effectiveness of that strain. A weight is assigned to the features according to the feedback score of the strain. For example, a feature that produced the most feedback score is given a highest weight. A matrix with these features is generated and filled in for these items. A weighted cosine similarity is found to recommend items on an ongoing basis (to accommodate for new strains that are added to the user's inventory).

The strains that are recommended as part of the experiment are stored in the user data 720 (FIG. 7) after the user selects what they are consuming. These user's selections are tracked so the application can remind the user after a set number of minutes to register their feedback with the feedback evaluator 710. The user data 720 associated with each experiment is used to incrementally learn the specific user's preferences and strain's effectiveness on their endocannabinoid system.

The user data 720 tracks the details of the user's consumption to date and the experiments associated with the collected data. This user data 720 may be encrypted and stored locally on a client device of the user. The user data may be aggregated, anonymized and published to backend server systems on a regular basis. It will be appreciated by those skilled in the art that this aggregated data will not contain any Protected Health Information (PHI) or Personally Identifiable Information (PII).

### Updating the Recommendation Based on User Feedback

As noted above, the feedback evaluator 710 provides a way to evaluate the effectiveness of a strain for a particular individual. Depending on the reason for a given experiment, the feedback process will be different. In sample configurations, the feedback process includes a video of the user to record facial expressions before and after consuming the strain. The feedback process may also include other questions that will be used to evaluate the effectiveness. These questions may be configured for different reasons in the backend systems and delivered to the user's device as needed. The actual feedback from these evaluations may be stored in the user data 720 as part of the corresponding experiment.

The feedback evaluated by the feedback evaluator 710 may be divided into three parts depending on the features:
1. THC Feedback System
2. Feedback for Medicinal Use and Effects
3. Feedback For Flavor and Aroma

The final evaluation score will be the sum of all the scores from all of these categories.

### 1. THC Feedback System

The THC content is directly proportional to the head high effect of a cannabis strain. A head high relates to increased energy levels, motivation, and rush of creative thinking often associated with consuming a sativa. This effect can be observed on one's facial features, post consumption. These changes in one's facial features can be used as a feedback to know if the recommended strain has less, more, or just the right THC content. Some of the most commonly observed facial feature changes are relaxation of facial muscles and increased blood pressure and heart rate resulting in redness of the eyes.

To capture the head high effect, user videos from before and after cannabis consumption are processed by a deep learning model to know the effectiveness of the THC. The videos need to be taken in a specific way and at a particular time post-consumption. These videos are sent to a deep learning model using a neural network that returns a score of THC effectiveness. This score is used as a feedback for the model to modify recommendations for the user.

The two videos taken need to meet following requirements for accurate results:
1. The user is required to take a picture of the user's face within a specified time frame just before cannabis consumption.
2. The user is required to take a picture of the user's face within a specific frame of, for example, 30 minutes post-consumption of a given amount of the strain.
3. The user is required to take both videos under similar conditions (e.g., same light and background).
4. The user has to consume a predefined amount of the strain.
5. The user is required to maintain a neutral face in both cases.

FIG. 9 is a diagram of a THC feedback system in a sample configuration. As shown in FIG. 9, the pictures taken before and after the consumption of the cannabis strain are provided to a trained neural network 900 that analyzes the before and after videos to generate a score. If the score is more than a threshold, the THC content of the strain is more than the user requires and a lower THC strain is recommended for the user in the next iteration. On the other hand, if the THC score is lower than the threshold, then the model may recommend strains of higher THC content in the next iteration. If the THC score is within a specified range of the threshold, then more strains of similar THC level may be recommended in the next iteration.

The neural network training may be done in a controlled environment with a specific quantity of strains of different THC levels of test subjects and observation of each experiment.

Since each experiment is conducted for a different reason, the user feedback based on effectiveness of the recommended strain for the particular reason needs to be considered. The user may rate the effectiveness of the strain on the particular medicinal case on a scale of, for example, 1-5. A similar user feedback may be recorded for the “Effect” feature (Relaxed, Focused, Giggly, etc.) which helps the model to recommend accurate strains for users seeking similar effects. In addition to producing the actual effects, this feedback feature will also convert the effects into a score. This can be done by maintaining a database table with scores assigned to each effect for a given reason.

For example, Table 1 below provides a score relating the effect and reason for consuming a particular strain:

TABLE 1

| Effect | Reason | Score |
|---|---|---|
| Relaxed | Sleep | 5 |
| Relaxed | Pain | 4 |
| Focused | Pain | 1 |

Feedback regarding the flavor and aroma is also useful data to retune the recommendation algorithm. This feedback may be aggregated across multiple users and used to retune the recommendation models. For the immediate scoring purposes, if the users highly rank the flavor and aroma, then a particular strain may be converted to a higher score (e.g., 5 on a scale of 1-5).

The Flavor and Aroma features of the database may be updated with the user responses. These entries get better as more users who have tested the particular strain provide feedback on the features. The users can voluntarily upvote or downvote a particular strain for its Aroma and Flavor independently. This data may be used to accurately recommend strains of the particular Aroma and Flavor for users seeking it. For example, a strain that receives a huge number of upvotes for a particular Aroma or Flavor can be recommended to users seeking that particular characteristic, and strain that receives a certain number of downvotes for a particular Aroma or Flavor can be avoided for users seeking for that particular characteristic.

FIG. 10 is a state diagram 1000 for tracking users and packages as the packages are delivered in a sample configuration. The state machine implementing the state diagram 1000 tracks the users as they are onboarded and the packages as they go through the delivery process.

As illustrated in FIG. 10, as soon as the user is created, the user will start with the Initiated state 1010. Users will be given a series of questions to understand their preferences. Once the users submit the responses to the questions, they will be placed in a Profiled state 1020. For all the users in the Profiled state 1020, a package will be assembled using the combination of the strain curator and delivery services. Once a package is assembled, the user is placed in the PackageReady state 1030. After the delivery service has created the shipment for a package, then the user is placed in the PackageDeliveryPending state 1040. When the package is actually delivered, the user is placed in the PackageReady state 1050. When the user is in the PackageReady state, a notification will be sent to the user to acknowledge. Once the user acknowledges, the user will be placed in the ReadyToConsume state 1060. Whenever the client application needs to replace a certain strain in the inventory, it can send a replacement order which will place the user back in the PackageReady state 1030, as illustrated.

FIG. 11 is a state diagram 1100 for tracking consumption of a product as users consume a strain for a specific reason in a sample configuration. The state machine implementing the state diagram 1100 tracks each experiment run as a user consumes a strain for a specific reason.

As illustrated in FIG. 11, the user either starts a new experiment or consumes a strain as part of an existing experiment. When consumption flow is initiated as part of an experiment, a recommendation of strains is created for the user and the state of that experimental run is set to RecommendationReady 1110. Once the list of recommendations is ready, the user may select the one they would like to try. When selected, the state of the experimental run is set to ConsumptionPending 1120. After the user consumes the selected strain, the state of the experimental run is set to FeedbackPending 1130. Depending on the strain consumed, the application may remind the user to register feedback after a certain interval. When the user comes back to the application, the feedback flow starts. When the feedback flow completes, the state of the experimental run is set to FeedbackComplete 1140, and a score associated with that run is stored in a database.

Thus, the user feedback is used to train the recommendation system based on the results of the respective experimental runs. The resulting updated recommendation system provides more accurate recommendations that are statically managed based on user research and eventually assembled dynamically when the user data (e.g., effectiveness of a given strain) has been obtained. Such methods of implementing user feedback provide an improved recommendation system that targets the needs of each consumer of the cannabis products.

### Sample Computer Platform for Implementing Cannabis Recommendation System

FIG. 12 illustrates one example of a computing device in the form of a computer 1200 that may include a processing unit 1202, memory 1204, removable storage 1206, and non-removable storage 1208 for implementing the processing features described above in a user client device and/or a server. Although the example computing device is illustrated and described as computer 1200, the computing device may be in different forms in different configurations. For example, the computing device may instead be a smartphone, a tablet, smartwatch, or other computing device including the same or similar elements as illustrated and described with regard to FIG. 12. Devices, such as smartphones, tablets, and smartwatches, are generally collectively referred to as mobile devices or user equipment. Further, although the various data storage elements are illustrated as part of the computer 1200, the storage may also or alternatively include cloud-based storage accessible via a network, such as the Internet or server-based storage.

Memory 1204 may include volatile memory 1210 and non-volatile memory 1212. Computer 1200 also may include, or have access to a computing environment that includes, a variety of computer-readable media, such as volatile memory 1210 and non-volatile memory 1212, removable storage 1206 and non-removable storage 1208. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 1200 may further include or have access to a computing environment that includes input interface 1214, output interface 1216, and a communication interface 1218. Output interface 1216 may include a display device, such as a touchscreen, that also may serve as an input device. The input interface 1214 may include one or more of a touchscreen, touchpad, mouse, keyboard, camera, one or more device-specific buttons, one or more sensors integrated within or coupled via wired or wireless data connections to the computer 1200, and other input devices.

The computer 1200 may operate in a networked environment using communication interface 1218 to connect to one or more remote computers. The remote computer may include a personal computer (PC), server, router, network PC, a peer device or other common network switch, or the like. The communication connection accessed via communication interface 1218 may include a Local Area Network (LAN), a Wide Area Network (WAN), cellular, Wi-Fi, Bluetooth, Zigbee, or other networks. According to one configuration, the various components of computer 1200 are connected with a system bus 1220.

Computer-readable instructions stored on a computer-readable medium are executable by the processing unit 1202 of the computer 1200, such as a program 1222. The program 1222 in some configurations comprises software that, when executed by the processing unit 1202, performs operations according to any of the configurations included herein. A hard drive, CD-ROM, and RAM are some examples of articles including a non-transitory computer-readable medium, such as a storage device. The terms computer-readable medium and storage device do not include carrier waves to the extent carrier waves are deemed too transitory. Storage can also include networked storage, such as a storage area network (SAN). Computer program 1222 may be used to cause processing unit 1202 to perform one or more methods or functions described herein.

It should be further understood that software including one or more computer-executable instructions that facilitate processing and operations as described above with reference to any one or all of steps of the disclosure may be provided in user portable electronic client devices to implement the features described herein. Alternatively, the software may be obtained and loaded into a server for implementing the features described herein through interaction with a user's client device. The software may be stored on a server for distribution over the Internet, for example.

Also, it will be understood by one skilled in the art that this disclosure is not limited in its application to the details of construction and the arrangement of components set forth in the above description or illustrated in the drawings. The configurations herein are capable of other configurations, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The components of the illustrative devices, systems and methods employed in accordance with the illustrated configurations may be implemented, at least in part, in digital electronic circuitry, analog electronic circuitry, or in computer hardware, firmware, software, or in combinations of them. These components also may be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program may be written in any form of programming language, including compiled or interpreted languages, and it may be deployed in any form, including as a stand-alone program or as a module, component, subroutine, or other unit suitable for use in a computing environment. A computer program may be deployed to be executed on one computer or on multiple computers at one site or distributed across multiple sites and interconnected by a communication network. Also, functional programs, codes, and code segments for accomplishing the systems and methods described herein may be easily construed as within the scope of the disclosure by programmers skilled in the art to which the present disclosure pertains. Method steps associated with the illustrative configurations may be performed by one or more programmable processors executing a computer program, code or instructions to perform functions (e.g., by operating on input data and generating an output). Method steps may also be performed by, and apparatus may be implemented as, special purpose logic circuitry, e.g., an FPGA (field programmable gate array) or an ASIC, for example.

The various illustrative logical blocks, modules, and circuits described in connection with the configurations disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random-access memory or both. The elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, compact disc ROM (CD-ROM), or digital versatile disc ROM (DVD-ROM). The processor and the memory may be supplemented by, or incorporated in, special purpose logic circuitry.

Those of skill in the art understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those skilled in the art may further appreciate that the various illustrative logical blocks, modules, circuits, and algorithm steps described in connection with the configurations disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. A software module may reside in random access memory (RAM), flash memory, ROM, EPROM, EEPROM, registers, hard disk, a removable disk, a CD-ROM, or any other form of storage medium known in the art. A sample storage medium is coupled to the processor such the processor may read information from, and write information to, the storage medium. In the alternative, the storage medium may be integral to the processor. In other words, the processor and the storage medium may reside in an integrated circuit or be implemented as discrete components.

As used herein, “machine-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., EEPROM), and any suitable combination thereof. The term “machine-readable medium” should be taken to include a single medium or multiple media (e.g., a centralized or distributed database, or associated caches and servers) able to store processor instructions. The term “machine-readable medium” shall also be taken to include any medium, or combination of multiple media, which is capable of storing instructions for execution by one or more processors, such that the instructions, when executed by one or more processors cause the one or more processors to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” as used herein excludes signals per se.

What is claimed:

1. A method of recommending a cannabis product to a user, comprising:
collecting data from the user regarding at least one of the user's lifestyle, preferences, medical conditions, or a video of the user performing a series of movements;
clustering the collected user data into a group with user data from other users to create a user profile;
creating a package of cannabis products based on the user profile;
providing the package of cannabis products to the user;
recommending to the user a strain of cannabis product within the package of cannabis products based on a purpose or occasion provided by the user for the cannabis consumption;
capturing pre-consumption and post-consumption video data of the user for a selected strain of cannabis product;
processing the pre-consumption and post-consumption video data of the user to measure effectiveness of the selected strain of the cannabis product for the given purpose or occasion; and
collecting feedback data from the user regarding the selected strain of cannabis product to feed back to fine tune the contents of the package of cannabis products recommended for the user profile.

2. The method of claim 1, wherein each group of clustered user data corresponds to a different curated box of strains of cannabis products.

3. The method of claim 1, wherein each group of clustered user data determines a design of the package and a marketing message associated with the package.

4. The method of claim 1, wherein the user profile includes at least one of favorite strains of cannabis product, reasons for consuming cannabis, answers to lifestyle related questions, or a video of the person doing predetermined movements.

5. The method of claim 1, further comprising aggregating, anonymizing, and periodically publishing the user data to a server.

6. The method of claim 1, wherein recommending to the user a strain of cannabis product within the package of cannabis products based on a purpose or occasion provided by the user for the cannabis consumption comprises:
creating a user vector that captures the user's interests relative to a predetermined number of unique properties of available strains of cannabis product;
creating a library matrix of strains of cannabis product that includes the available strains of cannabis product and features that associate each strain of cannabis product with its properties;
providing the user vector and the library matrix of cannabis product to an algorithm that creates a ranked list of strains of cannabis product that most closely match the user vector; and
providing at least one of the strains of cannabis product in the ranked list of strains in the package of cannabis products.

7. The method of claim 1, further comprising providing an input strain of cannabis product, computing a similarity score between the input strain and strains in the library matrix, and generating the ranked list with a predetermined number of most similar strains appearing first.

8. The method of claim 1, further comprising maintaining a list of strains of cannabis product to which the user currently has access, updating the list of strains of cannabis product as the user consumes strains of cannabis product, and updating a server for a cannabis product delivery system as the user consumes strains of cannabis product so that the cannabis product delivery system knows when and what to reorder.

9. The method of claim 1, wherein capturing pre-consumption and post-consumption video data of the user for a selected strain of cannabis product comprises taking a pre-consumption video of the user's face a first predetermined number of minutes before consuming the selected strain of cannabis product and taking a post-consumption video of the user's face a second predetermined number of minutes after consuming the selected strain of cannabis product.

10. The method of claim 9, wherein processing the pre-consumption and post-consumption video data of the user by a deep learning system to measure effectiveness of the selected strain of the cannabis product for the given purpose or occasion comprises extracting user data from facial cues in the pre-consumption video and post-consumption video.

11. The method of claim 1, wherein collecting feedback data from the user regarding the selected strain of cannabis product to feed back to fine tune the contents of the package of cannabis products recommended for the user profile comprises identifying a plurality of features of the strains of cannabis products that may be used to select a strain for user consumption, prioritizing a feature, ranking strains of cannabis products available for user consumption based on the prioritized feature, and assigning a weight to the prioritized feature according to a feedback score for the selected strain.

12. The method of claim 1, wherein the collected feedback data from the user regarding the selected strain of cannabis product comprises data relating to tetrahydrocannabinol (THC) content, medicinal use and effects, and flavor and aroma of the selected strain of cannabis product, further comprising determining a final evaluation score for the selected strain of cannabis product that is a sum of scores relating to the THC content, the medicinal use and effects, and the flavor and aroma of the selected strain of cannabis product.

13. The method of claim 12, further comprising determining a score of THC effectiveness of the selected strain of cannabis product by processing the pre-consumption and post-consumption video data of the user using a deep learning model to determine the THC effectiveness of the selected strain of cannabis product.

14. The method of claim 13, wherein determining the score of THC effectiveness comprises:
taking a pre-consumption picture of the user's face a first specified time before consumption of the selected strain of cannabis product;
consuming a predefined amount of the selected strain of cannabis product;
taking a post-consumption picture of the user's face a second specified time after consumption of the selected strain of cannabis product; and
providing the pre-consumption picture of the user's face and the post-consumption picture of the user's face to a trained neural network that analyzes the pre-consumption picture of the user's face and the post-consumption picture of the user's face to generate a THC effectiveness score.

15. The method of claim 12, further comprising determining a medicinal use and effects score based on effectiveness of the selected strain of cannabis product to treat a particular medicinal case or effect.

16. The method of claim 12, further comprising receiving feedback data from the user relating to flavor and aroma of the selected strain of cannabis product and determining a flavor and aroma score based on the received feedback data relating to flavor and aroma of the selected strain of cannabis product.

17. A system for recommending a cannabis product to a user, comprising:
a user profiler that collects data from the user regarding at least one of the user's lifestyle, preferences, medical conditions, or a video of the user performing a series of movements and clusters the collected user data into a group with user data from other users to create a user profile;
a strain curator that trains a machine learning model to create a package of cannabis products based on the user profile and to identify contents of a package of cannabis products recommended for the user based on the user profile; and
a delivery service that provides the package or cannabis products to the user and tracks a supply of cannabis products available to the user.

18. The system of claim 17, further comprising:
a strain recommender that recommends to the user a strain of cannabis product within the package of cannabis products based on a purpose or occasion provided by the user for the cannabis consumption; and
a feedback evaluator that captures pre-consumption and post-consumption video data of the user for a selected strain of cannabis product and processes the pre-consumption and post-consumption video data of the user to measure effectiveness of the selected strain of the cannabis product for the given purpose or occasion, wherein the feedback evaluator feeds back data from the user regarding the selected strain of cannabis product to fine tune the clustering by the user profiler and to train the machine learning model of the strain curator to update recommended contents of the package of cannabis products recommended for the user profile by the strain curator based on the pre-consumption and post-consumption video data of the user for the selected strain of cannabis product.

19. The system of claim 18, wherein the strain recommender creates a user vector that captures the user's interests relative to a predetermined number of unique properties of available strains of cannabis product, creates a library matrix of strains of cannabis product that includes the available strains of cannabis product and features that associate each strain of cannabis product with its properties, provides the user vector and the library matrix of cannabis product to an algorithm that creates a ranked list of strains of cannabis product that most closely match the user vector, and recommends inclusion of at least one of the strains of cannabis product in the ranked list of strains in the package of cannabis products.

20. The system of claim 18, wherein the feedback evaluator collects feedback data from the user regarding the selected strain of cannabis product, the feedback data comprising data relating to tetrahydrocannabinol (THC) content, medicinal use and effects, and flavor and aroma of the selected strain of cannabis product, the feedback evaluator further determining a final evaluation score for the selected strain of cannabis product that is a sum of scores relating to the THC content, the medicinal use and effects, and the flavor and aroma of the selected strain of cannabis product.
